SEQUENCE LISTING (2 sequences)

SEQ ID NO: 1 - DNA, 84 bp, Artificial Sequence
  Synthetic
  promoter (1)..(84)
ccctaatgggccaggcggcaggggttgagaggtaggggagatgggctctgagactataaagccagcgggggcccagcagccctc

SEQ ID NO: 2 - DNA, 363 bp, Artificial Sequence
  Synthetic
  promoter (1)..(363)
cagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctc

(19) United States
(12) Patent Application Publication
(10) Pub. No.: US 2022/0204988 A1
(43) Pub. Date: Jun. 30, 2022

(54) PROMOTER SEQUENCE AND RELATED PRODUCTS AND USES THEREOF

(71) Applicant: Oregon Health & Science University, Portland, OR (US)

(72) Inventors: Markus Grompe, Portland, OR (US); Sunghee Chai, Portland, OR (US)

(21) Appl. No.: 17/594,501

(22) PCT Filed: Apr. 24, 2020
(86) PCT No.: PCT/US2020/029918

§ 371 Date: Oct. 19, 2021

Related U.S. Application Data
(60) Provisional application No. 62/838,063, filed on Apr. 24, 2019.

(51) Int. Cl. C12N 15/86; C07K 14/62

(52) U.S. Cl. CPC C12N 15/86; C12N 2830/008; C12N 2750/14143; C07K 14/62

(57) ABSTRACT

The present disclosure provides ubiquitous short promoter elements capable of driving gene expression in multiple mammalian cell types of medical interest. Expression vectors incorporating the promoters are disclosed herein. Viral vectors, in particular, AAV vectors with capacity for larger transgenes than are possible with conventional promoters are disclosed herein. Methods of enhancing gene expression are disclosed herein.

### RELATED CASES

This application claims priority to U.S. Provisional Patent Application No. 62/838,063, which was filed on Apr. 24, 2019, which is hereby incorporated by reference in its entirety.

### STATEMENT REGARDING FEDERALLY SPONSORED RESEARCH

This invention was made with government support under Grant No. U01 DK089569 awarded by the National Institutes of Health. The government has certain rights in the invention.

### TECHNICAL FIELD

This disclosure relates generally to the field of biotechnology and in particular to a promoter sequence and related products and uses thereof.

### BACKGROUND

The most widely used strong ubiquitous promoters (CMV, CAG etc.) used in viral vectors are quite large (˜1,000 bp). This can be limiting in terms of the transgenes that can be expressed. For example, the AAV packaging limit is around 4.8 kb. After subtraction of the currently used promoters this leaves only around 3.7 kb for the transgene in a single-stranded AAV and only around 1.2 kb for self-complementary rAAVs. A need exists for a promoter that would allow for larger transgenes to be expressed.

### SUMMARY

Provided herein is a ubiquitous small promoter element capable of driving high yields of gene expression in multiple mammalian cell types of medical interest. Additionally, the promoter work well in human pancreatic endocrine cells (beta-cells and alpha-cells), as well as primary human hepatocytes. They are comparable in strength to the very strong ubiquitous promoters CMV and CAG.

The promoter can be used with plasmids and viral vectors. The promoter may allow insert transgenes with of about 4 kb in single stranded AAVs and about 1.6 kb in self-complementary AAVs. The size advantage is particularly striking for self-complementary vectors.

### BRIEF DESCRIPTION OF THE FIGURES

FIG. 1A depicts microscopy images of human embryonic stem (ES) cells differentiated to beta-like cells. Upper panels depict images of cells transduced with mRFP-expressing AAV-KP1 under the exemplary INS84 promoter. Lower panels depict cells that were not transduced with AAV. Left panels show bright-field microscopy images of ES-beta-like cells, while middle panels depict images of cells expressing green fluorescence protein (GFP) indicative of insulin expression. Right panels depict images of red fluorescence protein (RFP) suggesting transgene expression from the INS84 promoter.

FIG. 1B is a graphical representation of FACS analysis measuring RFP and C-peptide (c-pep), a peptide expressed in mature beta cells.

FIG. 2 depicts microscopy images of primary human pancreatic islets derived from cadavers transduced with mRFP-expressing AAV under the exemplary INS84 promoter (left panels) or the full length insulin promoter (right panels). Upper panels depict epifluorescence microscopy images of RFP, while lower panels depict bright-field microscopy images.

FIG. 3 are FACS analysis graphs of human islet cells transduced with exemplary AAV-INS84-mRFP. Left panel is a graph representing RFP expression in the total cell population tested. Right panel is a graph representing measurements of c-pep and glucagon (gcg) in RFP-positive cells. This indicates transgene expression in both alpha- and beta-cells.

FIG. 4 are graphical representations of FACS analysis of primary human islets cells transduced with exemplary AAV-INS84-mRFP (left panels) or AAV-INSx1-mRFP (right panels). Upper panels are graphs representing the measurements of side scatter versus RFP. Lower panels show quantitative values of cell populations.

FIG. 5 depicts microscopy images of human embryonic stem cell derived beta cells transduced with exemplary AAV-INS84-mRFP. From left to right are a bright-field microscopy image, depiction of GFP, and depiction of mRFP.

FIG. 6 are fluorescence microscopy images of human hepatocytes transduced with mRFP-expressing AAV under the exemplary INS84 promoter (left panel) or tdTomato expressing AAV under the CAG promoter (right panel).

FIG. 7 depicts epifluorescence microscopy images of RFP intensity in human embryonic kidney cells (left panel), mouse insulinoma cells (middle panel), and mouse alpha cells (right panel) transduced with exemplary AAV-INS84-mRFP.

### DETAILED DESCRIPTION

The present disclosure provides ubiquitous short promoter elements capable of driving gene expression in multiple mammalian cell types of medical interest. It may be comparable in strength to the very strong ubiquitous promoters CMV and CAG.

Disclosed herein is a promoter for transcription of genes and DNA elements in a cloning vector, including plasm ids and viral expression vectors. The example promoter (referred to herein as “INS84”) is composed of 84 base pairs derived from human insulin promoter and contains the core promoter TATA box and the upstream CAAT box region located upstream of the transcription start +1.

INS84 was tested for transcriptional activity in a recombinant adeno-associated virus (AAV) vector for potential use in gene therapy. Cells tested were human pancreatic islet cells, human embryonic stem (ES) cells and beta cells differentiated from ES cells, human hepatocytes and several immortal cell line cells. INS84 is active in all cells tested, categorizing it potentially as a strong universal promoter. It expressed the red fluorescent reporter transgene mRFP in all the cell types of human islets, including alpha, beta and other types. Furthermore, INS84 showed a higher activity than the full-length human insulin promoter (363 bp) in islets. Therefore, the INS84 promoter could be a useful tool for AAV-mediated gene expression and other expression vectors.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors.

“Identity”, as used herein, refers to the percent identity between two polynucleotide or two polypeptide moieties. The term “substantial identity”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. When referring to a polypeptide, or fragment thereof, the term “substantial identity” indicates that, when optimally aligned with appropriate gaps, insertions or deletions with another polypeptide, there is nucleotide sequence identity in about 90 to 100% of the aligned sequences. The term “highly conserved” means at least 80% identity, preferably at least 90% identity, and more preferably, over 97% identity. In some cases, highly conserved may refer to 100% identity. Identity is readily determined by one of skill in the art by, for example, the use of algorithms and computer programs known by those of skill in the art.

As described herein, alignments between sequences of nucleic acids or polypeptides are performed using any of a variety of publicly or commercially available Multiple Sequence Alignment Programs, such as “Clustal W”, accessible through Web Servers on the internet. Alternatively, Vector NTI utilities may also be used. There are also a number of algorithms known in the art that can be used to measure nucleotide sequence identity, including those contained in the programs described above.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

In particular embodiments, the promoter disclosed herein comprises a sequence of SEQ ID NO. 1, or a sequence having 80% identity to SEQ ID NO. 1; and the promoter excluding tissue-specific transcription factor binding site sequences. In some embodiments, the promoter has at least 85% sequence identity, at least 90% sequence identity, at least 95% sequence identity, at least 98% sequence identity, or at least 99% sequence identity to SEQ ID NO. 1. In some embodiments, the promoter comprises less than 200 nucleotides, less than 150 nucleotides, less than 100 nucleotides, less than 90 nucleotides, or having 84 nucleotides. In particular, the promoter comprises having 80 to 89 nucleotides, 90 to 99 nucleotides, 100 to 149 nucleotides, or 150 to 199 nucleotides.

An expression vector may comprise the promoter element described herein. The expression vector may be a plasmid or a viral vector such as an AAV vector, single stranded AAV, self-complementary AAV, adenovirus, Moloney murine sarcoma virus, murine stem cell virus, human immunodeficiency virus, Semliki Forest virus, Sindbis virus, Venezuelan equine encephalitis virus, Kunjin virus, West Nile virus, dengue virus, vesicular stomatitis virus, measles virus, Newcastle disease virus, vaccinia virus, cytomegalovirus, or coxsackievirus. The term “AAV vector” as used herein means any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. The term “AAV vector” may be used to refer to an AAV type viral particle (or virion) comprising a nucleic acid molecule encoding a protein of interest.

Additionally, the AAVs disclosed herein may be derived from various serotypes, including combinations of serotypes (e.g., “pseudotyped” AAV) or from various genomes (e.g., single-stranded or self-complementary). In particular embodiments, the AAV vectors disclosed herein may encode for desired proteins or protein variants.

In particular embodiments, the viral vector further comprises at least 3.7 kb of transgenes, at least 3.8 kb of transgenes, at least 3.9 kb of transgenes, or at least 4.0 kb of transgenes. In some embodiments, a capsid comprises a viral vector described herein.

The most widely used strong ubiquitous promoters (CMV, CAG etc.) used in rAAV vectors are quite large (1,000 bp). Given the packaging limit of rAAV, this is limiting in terms of the transgenes that can be expressed. AAV packaging limit is around 4.8 kb. After subtraction of the currently used promoters this leaves only around 3.7 kb for the transgene in a single-stranded AAV and only around 1.2 kb for self-complementary rAAVs. The promoter disclosed herein may allow insert transgenes with of about 4 kb in single stranded AAVs and about 1.6 kb in self-complementary AAVs along with two Inverted terminal repeats (ITRs) and a PolyA transcription termination signal sequence. In some embodiments, a single stranded AAV comprises from about 3.75 kb to about 4.6 kb of transgenes, about 3.8 kb to about 4.6 kb of transgenes, about 3.9 kb to about 4.6 kb of transgenes, or about 4.0 kb to about 4.6 kb of transgenes. In some embodiments, a self-complementary rAAV comprises from about 1.15 kb and about 2.0 kb of transgenes, about 1.2 kb and about 2.0 kb of transgenes, about 1.3 kb and about 2.0 kb of transgenes, about 1.4 kb and about 2.0 kb of transgenes, or about 1.5 kb and about 2.0 kb of transgenes.

Yet in some embodiments, the single-stranded AAV or the self-complementary rAAV further comprises the promoter described herein. In some embodiments, the single-stranded AAV or the self-complementary rAAV further comprising the promoter comprises a control element or control sequence.

Also described herein are methods of preparing a single-stranded AAV comprising from about 3.75 kb to about 4.6 kb of transgenes, about 3.8 kb to about 4.6 kb of transgenes, about 3.9 kb to about 4.6 kb of transgenes, or about 4.0 kb to about 4.6 kb of transgenes, the method comprising the use or insertion of the promoter described herein with a viral vector. Also described herein are methods of preparing a self-complementary rAAV comprising from about 1.15 kb and about 2.0 kb of transgenes, about 1.2 kb and about 2.0 kb of transgenes, about 1.3 kb and about 2.0 kb of transgenes, about 1.4 kb and about 2.0 kb of transgenes, or about 1.5 kb and about 2.0 kb of transgenes, the method comprising the use or insertion of the promoter described herein with a viral vector.

Methods of enhancing gene expression in a mammalian cell, the method comprising promoting expression of a particular gene with the promoter described herein are provided. The mammalian cell may be a human cell. In particular embodiments, the human mammalian cell may be a pancreatic endocrine cell, a pancreatic endocrine alpha cell, a pancreatic endocrine beta cell, a beta cell in human islets, a hepatocyte, or a primary human hepatocyte. Development of viral vectors has advanced the efficiency of viral transduction in a wide range of organs and tissues. Viral vectors can be a promising gene delivery tool in clinical applications, and could be developed to deliver genes to cells. Other means of delivering genes into cells are well known and described in the art, including various transfection techniques. Transfection and transduction methods can lead to transient or stable expression of DNA in host cells. Viral vectors may be an integrating vector or a non-integrating vector. For example, some lentivirus vectors are integration-proficient while some AAV vectors can persist in cells in an episomal form. Viral vectors may provide transient, short term to long term expression of transgenes. In some embodiments, a modified cell may comprise the promoter described herein.

### EXAMPLES

The following examples are for illustration only. In light of this disclosure, those of skill in the art will recognize that variations of these examples and other embodiments of the disclosed subject matter are enabled without undue experimentation.

### Example 1 Cell, Virus Vector, and Antibody Materials

Human pancreatic islets from non-diabetic donors were obtained from Integrated Islet Distribution Program (IIDP) at City of Hope. Human hepatocytes are obtained from Oregon Health and Science University by Dr. Bin Li in Grompe laboratory. Antibodies mouse-anti-glucagon (GCG) antibody and rat-anti-c-peptide (c-pep) were used to label alpha and beta cells, respectively. Protocols for human islet medium and medium for primary culture hepatocytes are well established in Grompe laboratory. The full-length insulin promoter (SEQ ID NO: 2) was obtained from the laboratory of Dr. Klaus Kaestner in University of Pennsylvania. AAV containing from CAG-tdTomato was produced by the OHSU viral vector core.

### Example 2 Cloning

AAV vectors containing INS84 (SEQ ID NO:1) promoter and the insulin full-length promoter, called INSx1, were cloned into a single stranded AAV (ssAAV) vector with the red fluorescent reporter transgene (mRFP). The INS84 sequence is located within the larger INSx1 sequence. The INS84 DNA fragment was cloned using synthetic oligomers of single stranded DNAs by forming duplex DNA followed by insertion into an ssAAV vector.

### Example 3 AAV Production

Standard methodology was used for AAV production. Briefly, HEK293 cells were cultured in a large quantity. Upon confluency, HEK293 cells were transfected by the polyethylenimine (PEI) method with three plasmids, ssAAV plasmids encoding promoter-m RFP, a helper plasm id from Adeno virus, and Rep/Cap plasm id pKP1 (Mark Kay laboratory). Five days after the transfection the newly packaged and released AAVs were collected from the cell culture medium. AAVs were then concentrated using PEG8000 and stored at −80° C.

### Example 4 Promoter Activity in Human Pancreatic Islets

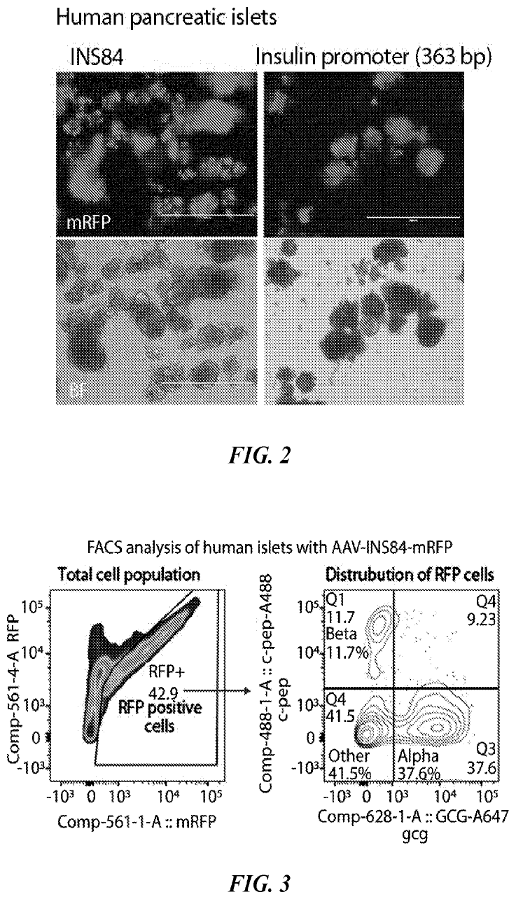

500 human islets per sample were mixed with AAV targeting multiplicity of infection (moi) of 10⁵. Transduction was carried out at 4° C. for 1 hour. Islets are then placed in a well of 24-well suspension culture plate containing human islet medium. Islets were then incubated at 37° C. in humidified CO2 incubator for 4 days. RFP expression of islets was recorded using an inverted epifluorescence microscope, shown in FIG. 2. Transgene mRFP is expressed from AAV under the INS84 (FIG. 2 left panels) and insulin 363 bp (INS×1) promoter.

Prior to FACS analysis of islet cells, islets were dissociated into individual cells using trypsin and fixed in 4% paraformaldehyde. Cells were then permeabilized with 0.1% Saponin/PBS for internalization of antibodies. Primary antibodies were then added to the dissociated islet cells. Rat-anti-C-peptide were used as a beta cell marker, to label the proinsulin cleavage product C-peptide. Alpha cells produce and secrete glucagon (GCG), which was used to label alpha cells with mouse-anti-GCG in this experiment. Secondary antibodies Alexa-488 and Alexa-647 were used to label anti-c-pep and anti-GCG, respectively. FACS analyses were performed using Symphony flow cytometer and data were analyzed using FlowJo software, shown in FIG. 3.

Data analyses were performed separating RFP positive cells from total cell populations and RFP cells into alpha and beta cell groups. Alpha and beta cells are two major types of cells in islets, making about 80% of the total population. Therefore, islet cells were grouped in three groups in our analysis, i.e., alpha, beta and the other cell group.

### Example 5—Comparison Analysis of AAV with INS84 and INS×1 (Insulin Full-Length Promoter) Activity

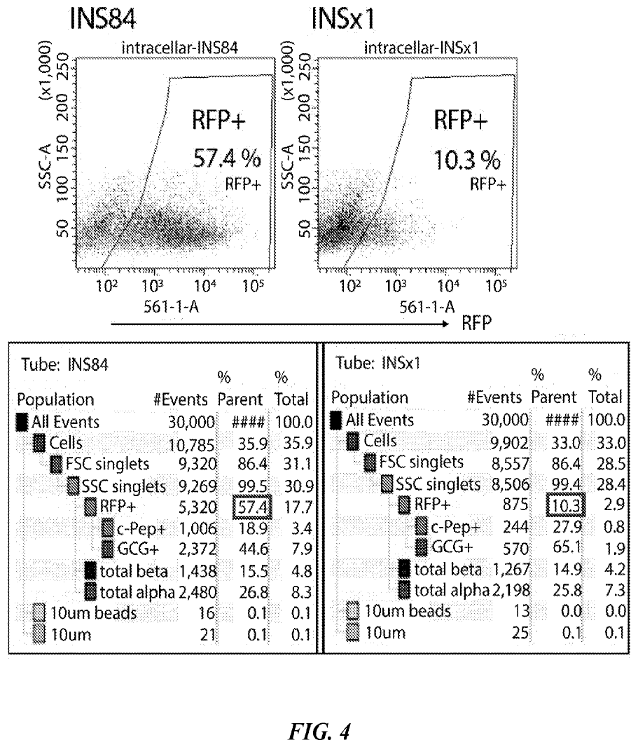

Human islets were transduced with AAV-INS84-mRFP or AAV-insulin full-length promoter (INS×1) with moi of 10⁵. After 4 days in culture, islets were dissociated and fixed for intracellular staining of alpha and beta cell marker. FACS analysis shows that AAV with the INS84 promoter transduces islet cell population more efficiently (57.4%) than AAV with INS×1 (10.3%) with a stronger intensity of RFP (see FIG. 4). Data are from a representative experiment. The intensity of RFP from the individual cells are shown in the RFP axis of the dot-plot (561-A). RFP intensity depends on the number of RFP protein molecules present in the cells. Thus, high RFP intensity reflects high number of mRNA molecules and accordingly indication of strong promoter. The most of RFP cells are detected scales between 10³ and 10⁴ in INS84 cells and between 10² and 10³ in INS×1 cells. These data suggest that INS84 activity is about 10 times higher than that of INS×1.

### Example 6 INS84 Promoter Activity in Human ES Cells

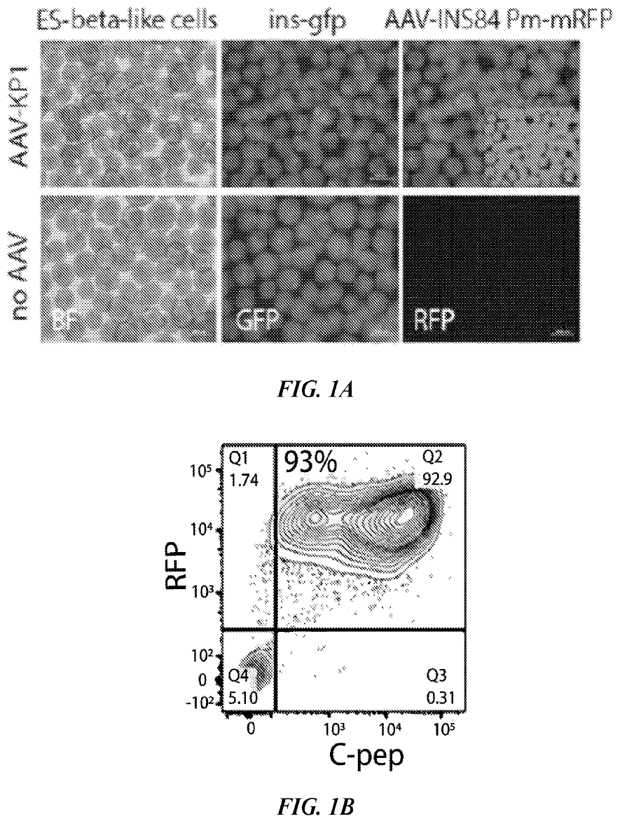

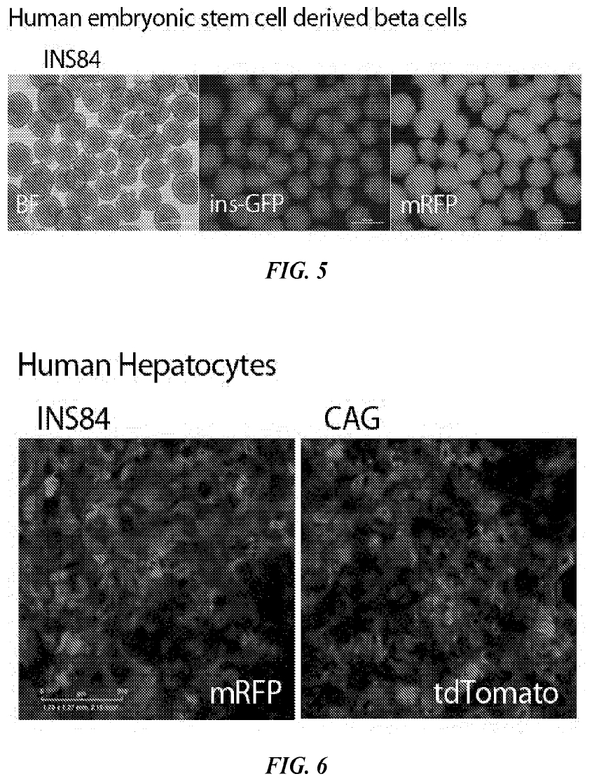

AAV transduction in ES cells were performed by our collaborators, Dr. Matthias Hebrok and Dr. Youngjin Kim at the University of California, San Francisco. The ES cells have genomic integration of the GFP gene in downstream of the insulin promoter. Insulin gene is expressed only in beta cells. Upon acquiring identity of beta cell character during the cell differentiation, the insulin promoter becomes active and thus GFP is expressed from these cells. Briefly, AAV testing was performed as follows. ES cells were enriched, and spheres were incubated for 20 days in a beta cell differentiation medium. The fully differentiated beta cells were sorted by FACS to select GFP expressing cells. These cells (eBeta cells) were then transduced with AAV-INS84-mRFP with moi 10⁵. Four days after transduction, cells were analyzed by FACS following fixation, antibody labelling with anti-C-pep and anti-GCG, shown in FIG. 5. Human embryonic stem (ES) cells were differentiated to beta-like cells. GFP gene is expressed from the insulin gene promoter from the chromosome only in beta cells. Transduction of AAV with INS84 promoter expresses mRFP in all eBeta cells with strong intensity. Also shown are eBeta cells transduced with mRFP-expressing AAV KP1 under the INS84 promoter, where the majority of cells are RFP positive (see FIGS. 1A and 1B).

### Example 7 INS84 Promoter Activity in Human Hepatocytes

Tests with human hepatocytes were performed by Dr. Bin Li in Grompe laboratory. Following the laboratory standard protocol, the donor liver cells were dissociated and plated in wells of 24-well plates. AAVs, AAV-INS84-mRFP and AAV-CAG-tdTomato, are added to the wells to moi of 10⁵. To monitor and record red fluorescence from the transgenes during the cell growth, the culture plate was placed in a humidified CO2 incubator at 37° C. which is equipped with an automated microscopy (incubator microscopy) in Advanced light microscopy core at OHSU. Images from the day 5 culture were shown in the FIG. 6. The INS84 promoter was compared to CAG promoter in a primary culture of human hepatocytes. AAV with INS84 promoter expresses mRFP and AAV with CAG promoter expresses tdTomato. Expression levels were equally strong.

### Example 8 INS84 Promoter Activity in Cell Lines

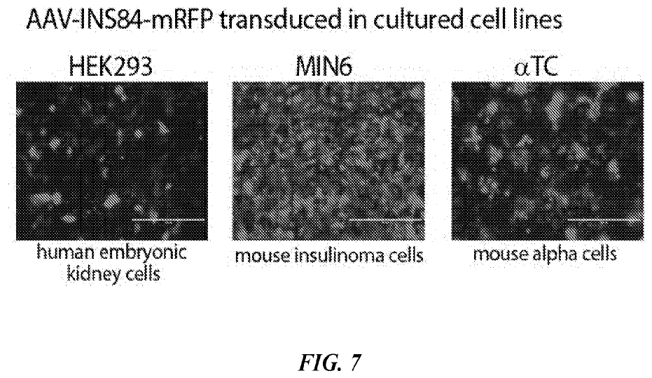

Cell lines were grown in their respective culture conditions. One day before the AAV transduction cells were plated in 12-well to 50% cell density. For transduction AAV-INS84-mRFP was directly added to the culture medium to moi of 10⁵. Images were taken using an inverted epifluorescence microscope at days between 4 and 6, shown in FIG. 7. Cell line cells were transduced with AAV-INS84-mRFP. INS84 is active in all cells tested, albeit with different intensities of RFP. The differences could be a result from availability of viral receptors on the cell surface or differences in promoter activity in different cell types, which is commonly observed with other known promoters.

1. A promoter comprising:
a sequence of SEQ ID NO. 1, or a sequence having 80% identity to SEQ ID NO. 1; and the promoter excluding tissue-specific transcription factor binding site sequences.

2. The promoter of claim 1 having at least 85% sequence identity, at least 90% sequence identity, at least 95% sequence identity, at least 98% sequence identity, or at least 99% sequence identity to SEQ ID NO. 1.

3. The promoter of claim 1 comprising less than 200 nucleotides, less than 150 nucleotides, less than 100 nucleotides, less than 90 nucleotides, or having 84 nucleotides.

4. An expression vector comprising the promoter of claim 1.

5. The expression vector of claim 4, wherein the expression vector comprises a plasmid, an AAV vector, single stranded AAV, self-complementary AAV, adenovirus, Moloney murine sarcoma virus, murine stem cell virus, human immunodeficiency virus, Semliki Forest virus, Sindbis virus, Venezuelan equine encephalitis virus, Kunjin virus, West Nile virus, dengue virus, vesicular stomatitis virus, measles virus, Newcastle disease virus, vaccinia virus, cytomegalovirus, or coxsackievirus.

6. The expression vector of claim 4, further comprising at least 3.7 kb of transgenes, at least 3.8 kb of transgenes, at least 3.9 kb of transgenes, or at least 4.0 kb of transgenes.

7. A capsid comprising a viral expression vector of claim 4.

8. A single-stranded AAV comprising from about 3.75 kb to about 4.6 kb of transgenes, about 3.8 kb to about 4.6 kb of transgenes, about 3.9 kb to about 4.6 kb of transgenes, or about 4.0 kb to about 4.6 kb of transgenes.

9. A self-complementary recombinant adeno-associated virus (rAAV) comprising from about 1.15 kb and about 2.0 kb of transgenes, about 1.2 kb and about 2.0 kb of transgenes, about 1.3 kb and about 2.0 kb of transgenes, about 1.4 kb and about 2.0 kb of transgenes, or about 1.5 kb and about 2.0 kb of transgenes.

10. The single-stranded AAV of claim 8, further comprising a promoter comprising:
a sequence of SEQ ID NO. 1, or a sequence having 80% identity to SEQ ID NO. 1; and the promoter excluding tissue-specific transcription factor binding site sequences.

11. A method of preparing the single-stranded AAV of claim 8, the method comprising the use or insertion of a promoter with a viral vector, the promoter comprising:
a sequence of SEQ ID NO. 1, or a sequence having 80% identity to SEQ ID NO. 1; and the promoter excluding tissue-specific transcription factor binding site sequences.

12. A method of enhancing gene expression in a mammalian cell, the method comprising promoting expression of a particular gene with the promoter of claim 1.

13. The method of claim 12, wherein the mammalian cell is a human cell.

14. The method of claim 12, wherein the cell is a human pancreatic endocrine cell.

15. The method of claim 12, wherein the cell is a human pancreatic endocrine alpha cell.

16. The method of claim 12, wherein the cell is a human pancreatic endocrine beta cell.

17. The method of claim 12, wherein the cell is a beta cell in human islets.

18. The method of claim 12, wherein the cell is a human hepatocyte.

19. The method of claim 12, wherein the cell is a primary human hepatocyte.

20. A modified cell comprising the promoter of claim 1.
